(19) United States
(12) Patent Application Publication
(10) Pub. No.: US 2007/0073556 A1
(43) Pub. Date: Mar. 29, 2007

(54) System and method for coordinating examination scheduling

(75) Inventors: Denny Lau, Sunnyvale, CA (US); Raghav Raman, Cupertino, CA (US); Lawrence Chow, Portland, OR (US); Sandip Biswal, Stanford, CA (US); Andrew Sorrentino, Northbrook, IL (US); Eric Feingold, Philadelphia, PA (US); Vijaykalyan Yeluri, Sunnyvale, CA (US)

Correspondence Address: MCANDREWS HELD & MALLOY, LTD, 500 WEST MADISON STREET, SUITE 3400, CHICAGO, IL, 60661, US

(73) Assignee: General Electric Co.

(21) Appl. No.: 11/234,698

(22) Filed: Sep. 23, 2005

(51) Int. Cl. G06Q 10/00; A61B 5/00

(52) U.S. Cl. 705/2; 600/300

(57) ABSTRACT

Certain embodiments of the present invention provide a method of coordinating scheduling including: evaluating patient associated information corresponding to a patient having a scheduled examination; evaluating healthcare service provider associated information corresponding to at least one healthcare service provider; predicting an efficiency for one of the at least one healthcare service provider performing the scheduled examination based at least in part on the patient associated information and the healthcare service provider associated information; and adjusting a state of a coordination indicator based at least in part on the efficiency.

### BACKGROUND OF THE INVENTION

Embodiments of the present invention relate generally to coordinating scheduling between an examiner and a patient. Particularly, certain embodiments relate to coordinating scheduling between an available radiologist and a scheduled patient by associating an indicia with a scheduled patient.

A healthcare enterprise, such as a hospital or clinic, may be a complicated and dynamic place. Every day, the enterprise must provide services for a potentially large number of scheduled patients. At the same time, staffing of healthcare service providers may change from day-to-day. The healthcare enterprise staffing administration, therefore, may be faced with the task of matching up available service providers with the needs of scheduled patients. Note, it should be understood that individuals who provide healthcare services and administration may either be employees of a healthcare enterprise, or may be independent contractors or the like. The legal status of a particular healthcare service provider or administrator is not relevant to the present invention.

Certain healthcare service providers may be in high demand and relatively uncommon. For example, specialists, such as pediatric neurologists and ophthalmologists may be in high demand and relatively uncommon. For such healthcare service providers, it may be impractical for a healthcare enterprise to staff more than one individual having a particular specialty per shift. There may, therefore, be only one service provider at an enterprise for patients requiring a particular specialty.

However, other types of healthcare service providers may be more common. For example, nurse practitioners may be staffed in larger numbers. Certain types of physicians may also be staffed in larger numbers, such as internists, pathologists, anesthesiologists, and radiologists. The services provided by such healthcare workers may be relatively interchangeable from the perspective of a patient. For example, if four radiologists are scheduled to work a shift, any of the four radiologists may competently analyze a patient's medical image data. There may, however, be various different sub-specializations within a certain type of staffed healthcare service provider. For example, each of the four radiologists may have enhanced skills and ability to provide service in different sub-specialization areas. Examples of radiology sub-specialization include breast imaging, ultrasonography, and pediatric radiology.

Healthcare enterprises, such as hospitals or clinics, include clinical information systems, such as hospital information systems (HIS) and radiology information systems (RIS), and storage systems, such as picture archiving and communication systems (PACS). Information stored may include patient medical histories, imaging data, test results, diagnosis information, management information, examination reports, and/or scheduling information, for example. The information may be centrally stored or divided at a plurality of locations. Healthcare service providers may desire to access patient information or other information at various points in a healthcare workflow. For example, during scheduling, healthcare service providers may access patient information, such as a patient's medical history and examination reports, stored in a clinical information system. Healthcare service providers may review patient information. Also, healthcare service providers may edit patient information or enter new information, such as history, diagnostic, scheduling, or treatment information, into a clinical information system.

A PACS workstation may be provided to access information in one or more clinical information systems, such as HIS, RIS, or PACS. A PACS workstation may be used, for example, to display patient scheduling information. Scheduling information may be entered in a RIS. The RIS may communicate the scheduling information to a PACS via HL7 messages. Upon the receipt of these HL7 messages, a PACS may update a database that includes patient scheduling information.

Healthcare service providers may use a PACS workstation to retrieve information relating to scheduled patients for a given shift or time period. For example, a radiologist may use a PACS workstation to view a list of scheduled exam list for that day. A healthcare service provider may select a subset of examinations from a full list of scheduled patients for examinations. For example, if three radiologists are scheduled during a shift, a single radiologist may select one-third of examinations from all of the patient examinations scheduled during a shift. Once an exam is acquired and has passed a quality assurance check, it falls into a queue that a radiologist uses to retrieve unread exams. The radiologist selects an exam from the queue and opens the exam for viewing and interpretation. Once the radiologist has finished interpreting the exam by dictating a report, the radiologist changes the exam status and the exam drops off the reading queue.

A healthcare service provider may be able to perform certain examinations more efficiently than others. For example, a radiologist with a sub-specialization in breast imaging may be able to more rapidly analyze mammography images than a radiologist who does not have a breast imaging sub-specialization. While any qualified radiologist may provide effective service, a radiologist with the breast imaging sub-specialization may provide service more efficiently, for example. A healthcare service provider may desire to examine only those scheduled patients or examinations which he or she may perform efficiently. Also, a healthcare service provider may desire to decline examinations that other healthcare service providers may perform more efficiently.

Various types of patient information may be helpful in predicting whether a particular healthcare service provider may efficiently perform an examination. This helpful information may be accessible through a PACS workstation. However, conventional systems may not effectively communicate helpful information to a healthcare service provider who is deciding whether or not to perform an examination. Similarly, conventional systems may not correlate helpful information with scheduled patient exams.

Thus, there is a need for methods and systems that communicate helpful information to a healthcare service provider who is deciding whether to perform an examination of a scheduled patient. There is a need for methods and systems that enable a healthcare service provider to select efficiently performable examinations. Additionally, there is a need for methods and systems that enable a healthcare service provider to defer an examination to another healthcare service provider who may perform the examination efficiently.

### BRIEF SUMMARY OF THE INVENTION

Certain embodiments of the present invention provide a method of coordinating scheduling including: evaluating patient associated information corresponding to a patient having a scheduled examination; evaluating healthcare service provider associated information corresponding to at least one healthcare service provider; predicting an efficiency for one of the at least one healthcare service provider performing the scheduled examination based at least in part on the patient associated information and the healthcare service provider associated information; and adjusting a state of a coordination indicator based at least in part on the efficiency. In an embodiment, the healthcare service provider includes a radiologist. In an embodiment, the healthcare service provider associated information includes at least one of: a logon presence of the at least one healthcare service provider; an identity of a staffed the at least one healthcare service provider; a specialty of the staffed the at least one healthcare service provider; an availability of the at least one healthcare service provider; and an experience of the staffed the at least one healthcare service provider. In an embodiment, at least a portion of the method is performable by a Picture Archiving and Communications System. In an embodiment, the efficiency is based at least in part on a prior examination of the patient having been performed by an available one of the at least one healthcare service provider. In an embodiment, the patient associated information includes at least one of: information that the patient is a returning patient; a date of a prior examination of the patient; information that the patient is scheduled to have an additional follow-up examination; a name of a healthcare service provider who participated in the prior examination; a name of a healthcare service provider who approved the prior examination; a name of healthcare service provider associated with the prior examination; a type of the scheduled examination; a purpose of the scheduled examination; a modality of the scheduled examination; and a number of images in the scheduled examination. In an embodiment, the state of the coordination indicator corresponds to at least one of: a suggestion for one of the at least one healthcare service provider to perform the scheduled examination; a suggestion for other of the at least one healthcare service provider to perform the scheduled examination; and no suggestion for any of the at least one healthcare service provider to perform the scheduled examination. In an embodiment, the method further includes assigning the at least one healthcare service provider to perform the scheduled examination based at least in part on the efficiency. In an embodiment, the method further includes notifying at least one party based at least in part on the efficiency, the at least one party including at least one of: the patient; the at least one healthcare service provider; a healthcare service administrator; an insurance provider; an insurance administrator.

Certain embodiments of the present invention provide a system for coordinating patient examination scheduling including: patient associated information corresponding to a patient having a scheduled examination; healthcare service provider associated information corresponding to at least one healthcare service provider scheduled to work at the time of the scheduled examination; a prediction module capable of receiving the patient associated information and the healthcare service provider associated information, the module capable of predicting an efficiency of having one of the at least one healthcare service provider perform the scheduled examination based at least in part on the patient associated information and the healthcare service provider associated information; and an application software capable of displaying a coordination indicator based at least in part on the efficiency. In an embodiment, the healthcare service provider includes a radiologist. In an embodiment, the efficiency is based at least in part on a correspondence between the patient associated information and the healthcare service provider associated information. In an embodiment, the healthcare service provider associated information includes at least one of: an identity of an available healthcare service provider, and the patient associated information includes a prior examination approved by the available healthcare service provider. In an embodiment, the available healthcare service provider approved the prior examination. In an embodiment, at least a portion of the prediction module is executable on a Picture Archiving and Communication System. In an embodiment, the coordination indicator corresponds to at least one of: a suggestion for one of the at least one healthcare service provider to perform the scheduled examination; a suggestion for other of the at least one available healthcare service provider to perform the at least one scheduled examination; and no suggestion for any of the at least one healthcare service provider to perform the scheduled examination. In an embodiment, healthcare service provider associated information includes at least one of: a logon presence of the at least one healthcare service provider; an identity of a staffed the at least one healthcare service provider; a specialty of the staffed the at least one healthcare service provider; an availability of the at least one healthcare service provider; and an experience of the staffed the at least one healthcare service provider. In an embodiment, the patient associated information includes at least one of: information that the patient is a returning patient; a date of a prior examination of the patient; information that the patient is scheduled for an additional follow-up examination; a name of a healthcare service provider who participated in the prior examination; a name of a healthcare service provider who approved the prior examination; a name of healthcare service provider associated with the prior examination; a type of the scheduled examination; a purpose of the scheduled examination; a modality of the scheduled examination; and a number of images in the scheduled examination.

Certain embodiments of the present invention provide a computer-readable storage medium including a set of instructions for a computer, the set of instructions including: at least one evaluation routine for evaluating patient associated information corresponding to a patient having a scheduled examination and for evaluating healthcare service provider associated information corresponding to at least one healthcare service provider; a prediction routine for predicting an efficiency for one of the at least one healthcare service provider performing the scheduled examination based at least in part on the patient associated information and the healthcare service provider associated information; and an adjustment routine for adjusting a state of a coordination indicator based at least in part on the efficiency. In an embodiment, the healthcare service provider includes a radiologist. In an embodiment, the healthcare service provider associated information includes at least one of: a logon presence of the at least one healthcare service provider on a Picture Archiving and Communications System; an identity of a staffed the at least one healthcare service provider; a specialty of the staffed the at least one healthcare service provider; an availability of the at least one healthcare service provider; and an experience of the staffed the at least one healthcare service provider. In an embodiment, the efficiency is based at least in part on a prior examination of the patient having been performed by an available one of the at least one healthcare service provider. In an embodiment, the state of the coordination indicator corresponds to at least one of: a suggestion for one of the at least one healthcare service provider to perform the scheduled examination; a suggestion for other of the at least one healthcare service provider to perform the scheduled examination; and no suggestion for any of the at least one healthcare service provider to perform the scheduled examination. In an embodiment, the patient associated information includes at least one of: information that the patient is a returning patient; a date of a prior examination of the patient; information that the patient is scheduled for an additional follow-up examination; a name of a healthcare service provider who participated in the prior examination; a name of a healthcare service provider who approved the prior examination; a name of healthcare service provider associated with the prior examination; a type of the scheduled examination; a purpose of the scheduled examination; a modality of the scheduled examination; and a number of images in the scheduled examination.

### BRIEF DESCRIPTION OF SEVERAL VIEWS OF THE DRAWINGS

FIG. 1 shows a block diagram of various clinical information systems networked in a healthcare enterprise.

FIG. 2 shows a block diagram of a picture archiving and communication system according to an embodiment of the present invention.

FIG. 3 shows a block diagram of a PACS workstation according to an embodiment of the present invention.

FIG. 4 shows a representation of a view of an application program for scheduling coordination according to an embodiment of the present invention.

FIG. 5 shows a block diagram of information useable by a prediction module to predict an efficiency according to an embodiment of the present invention.

FIG. 6 shows a flowchart of a method for adjusting a state of coordination indicator in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

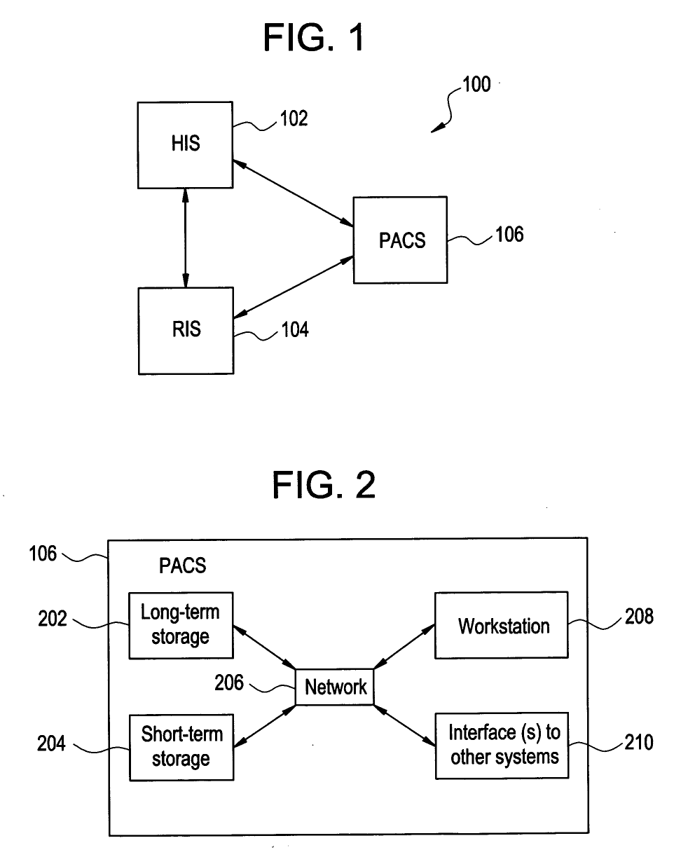

FIG. 1 shows a block diagram of various clinical information systems networked in a healthcare enterprise. Various clinical information systems may include a Hospital Information System (“HIS”) 102, a Radiological Information System (“RIS”) 104, and a Picture Archiving and Communication System (“PACS”) 106. A RIS 104 may include a patient scheduling module. The patient scheduling module may coordinate and schedule examinations according to available, allotted time slots for image acquisition systems. Different types of exams may have different time allocations because some exams are more time consuming than others.

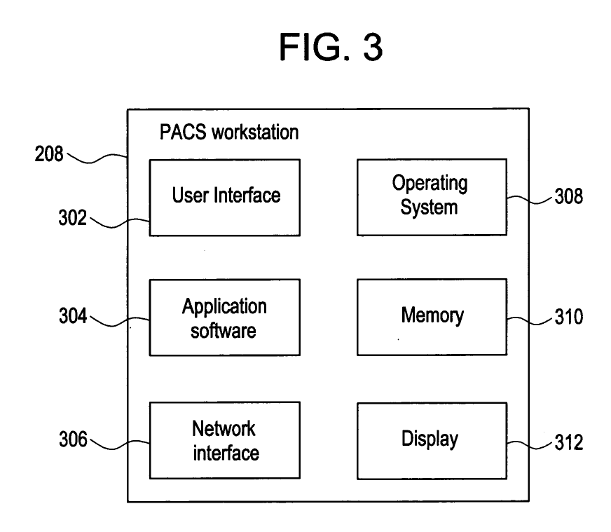

FIG. 2 shows a block diagram of a PACS 106 according to an embodiment of the present invention. A PACS 106 may include a long-term storage 202, a short-term storage 204, a workstation 208, and interface(s) to other systems 210. Each of these components may communicate each other, or outside the PACS 106 through a network 206. A network may be a wired, wireless, or optical network, or a combination thereof. A long-term storage 202 may include, for example, a PACS archive. A long-term storage 202 may be a separate PACS device, and may include nonvolatile digital storage media. A short-term storage 204 may be a separate PACS device, or may be a part of another PACS device, such as a PACS workstation 208, for example. A short-term storage 204 may include digital storage media, such as RAM, for example. Interface(s) to other systems 210 may include, for example, an interface to a RIS 104 (shown in FIG. 1) and/or an interface to a HIS 102 (shown in FIG. 1). PACS 106 may include additional components not depicted in FIG. 2, such as routers, repeaters, hubs, buffers, amplifiers, splitters, and the like.

FIG. 3 shows a block diagram of a PACS workstation 208 according to an embodiment of the present invention. PACS workstation 208 may be a personal computer, laptop, desktop, workstation, dumb terminal, thin client, or the like. A PACS workstation 208 may include a user interface 302, application software 304, network interface 306, operating system 308, memory 310, and display 312. A user may interact with PACS workstation 208 through a user interface 302. A user interface 302 may include, for example, a mouse and keyboard. A user, such as a healthcare service provider, may logon to the PACS workstation 208 through user interface 302. PACS workstation 208 may have operating system 308 that enables execution of application software 304. Operating system 308 may also facilitate interaction between user interface 302, application software 304, network interface 306, memory 310, and display 312. Memory 310 may include digital memory, such as RAM, ROM, flash memory, hard disk, optical disk, magnetic disk, or the like. Memory 310 may be used to temporarily store information retrieved through network interface 306 and accessed by application software 304. For example, memory 310 may be used to store patient records that are retrieved from a PACS long-term storage (shown in FIG. 2) through network interface 306. Similarly, memory 310 may be used to temporarily edit, add, remove, or update information with application software 304 to be transferred through network interface 306. For example, memory 310 may be used to update patient records that are retrieved from a PACS long-term storage (shown in FIG. 2) through network interface 306. Application software 304 may be used to edit, add, remove, or update information associated with patient records. The altered or new information may later be transferred to PACS long-term storage (shown in FIG. 2) through network interface 306. Application software 304 may be imaging software, scheduling software, exam status tracking software, patient history software, for example.

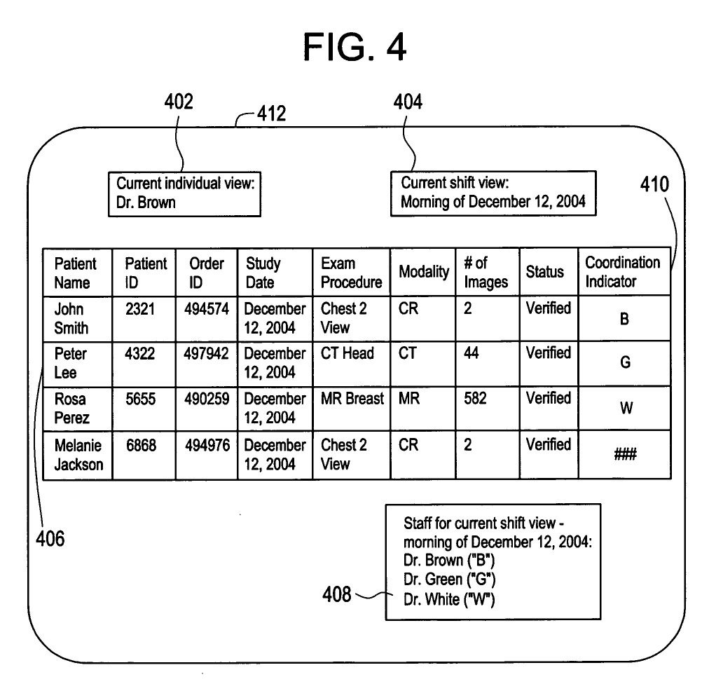

FIG. 4 shows a representation of a view of an application program for scheduling coordination according to an embodiment of the present invention. An application program may be software, and an application program may be implemented as a set of instructions on a computer readable medium, for example. The instructions may be executable on a processor or controller, for example, including a client, a server, a workstation, a laptop, a desktop, a microprocessor, a plurality of microprocessors, or the like. A display 412 may be similar to display 312 (shown in FIG. 3) on a PACS workstation 208 (shown in FIG. 3). Display 412 shows a representation of a view that may be generated by scheduling coordination software. The scheduling coordination software may be similar to one or more types of application software 304 (shown in FIG. 3) that may operate on a PACS workstation 208 (shown in FIG. 3). The scheduling coordination software may provide graphical and textual information to display 412. The scheduling coordination software view may include a current individual view caption 402, a current shift view caption 404, scheduled patient information 406, staff for current shift view caption 408, and coordination indicator 410.

The current individual view caption 402 may display a name of a particular healthcare service provider. For example, the current individual view caption 402 may display a name of a radiologist. The current individual view caption 402 may indicate that the scheduling coordination software is displaying information that is customized, tailored, or intended for view by a particular healthcare service provider. The current individual view caption 402 may display the name of a healthcare service provider who is currently using the PACS workstation 208. There may be a variety of ways to recognize who is currently using a PACS workstation 208. For example, current individual view caption 402 may show the name of a user who is currently logged onto a PACS workstation or logged onto application software 304. The current individual view caption 402 may be changeable by the user. For example, the user may select a different current individual view caption 402 through a drop-down menu, or through a software option or preference. Information presented in one individual view may differ from information presented in another individual view. A user may have an option to customize a particular individual view. A user may also be able to customize multiple individual views. The current individual view caption 402 need not display particular words, such as “current individual view.” Also, the current individual view caption 402 may be omitted. If current individual view caption 402 is omitted, the scheduling coordination software may still be able to display various individual views resulting in differing displays of information. For example, different information may be displayed based on who is logged onto the PACS workstation, even if there is no current individual view caption 402.

The current shift view caption 404 may indicate that the information displayed on the screen corresponds to a particular shift. It should be understood that a shift may be a period of time that one or more individuals are scheduled to work. A shift may be a whole shift, or may only be a portion thereof. For example, the current shift view caption 404 may show the current shift, or may show the next scheduled shift for a particular healthcare service provider. A shift may be chosen based on a current date/time, or may be selectable for a user. The current shift view caption 404 may indicate, for example, that the scheduling coordination software is displaying information corresponding to the morning portion of the present day. The current shift view caption 404 need not display particular words, such as “current shift view.” The current shift view caption 404 may be customizable, or may be omitted. If current shift view caption 404 is omitted, it is understood that the scheduling coordination software may be able to display different shift views resulting in differing displays of information. For example, different information may be displayed based on the current date/time, even if there is no current shift view caption 404.

Scheduled patient information 406 may include a variety of information corresponding to scheduled patients. As discussed, scheduled patient information 406 may show a variety of information based on an individual view (that may be displayed in the current individual view caption 402). Similarly, scheduled patient information 406 may show a variety of information based on a shift view (that may be displayed in the current shift view caption 404). Scheduled patient information 406 may include information corresponding to a plurality of scheduled patients. For example, scheduled patient information 406 may display the following information for each patient: patient name, patient ID, order ID, study date, exam procedure, modality, number of images, and status. The scheduling coordination software may display only a portion of patient information corresponding to scheduled patients. For example, scheduling coordination software may be able to retrieve or associate patient data from a variety of sources, such as a PACS long-term storage 202 (shown in FIG. 2) and a PACS short-term storage 204 (shown in FIG. 2). Scheduling coordination software may display only that information that may be considered relevant. As another option, scheduling coordination software may be customizable to display a variety of patient information in a variety of configurations. For example, either a user or a system administrator may be able to customize the display of the scheduling coordination software.

Staff for current shift view caption 408 may show a list of healthcare service providers who are scheduled to work during a particular shift. For example, staff for current shift view caption 408 may show all providers who are scheduled to work during the shift indicated in current shift view caption 404. Staff for current shift view 408 may show all of a certain type of healthcare service providers who are scheduled to work during a particular shift. For example, staff for current shift view 408 may show all radiologists scheduled to work during a particular shift. The staff for current shift view caption 408 need not display particular words, such as “staff for current shift view.” Staff for current shift view 408 may be omitted. If staff for current shift view caption 408 is omitted, the scheduling coordination software may still be able to track which staff are scheduled for the currently displayed shift, and may still be able to use this information for processing or for conveying to a user.

Coordination indicator 410 may display one of a variety of indicators in association with one or more scheduled patients. A coordination indicator 410 may indicate to a healthcare service provider whether he or she should perform an examination of a patient. For example, a coordination indicator 410 may indicate to a radiologist whether he should examine a particular patient's radiological images. Coordination indicator 410 may also communicate to a healthcare service provider that he or she may perform a particular exam more efficiently than other staffed healthcare service providers. The coordination indicator 410 may have a plurality of states. For example, coordination indicator 410 may have two, three, or more states.

For example, in an embodiment where the coordination indicator 410 has two states, the first state may indicate to a radiologist that he may efficiently perform an examination. A second state may indicate to a radiologist that he may not perform a particular examination with efficiency. As another example, in an embodiment where the coordination indicator 410 has three states, the first state may indicate to a radiologist that he may efficiently perform an examination. A second state may indicate to a radiologist that another staffed radiologist may perform the examination more efficiently. A third state may indicate that there may be no efficiency advantage among staffed radiologists.

As another example, in an embodiment the coordination indicator 410 has more than three states. An example of a coordination indicator 410 having more than three states is shown in FIG. 4. The coordination indicator column contains four different states—“B”, “G”, “W”, and “###”—associated with four different patients—John Smith, Peter Lee, Rosa Perez, and Melanie Jackson—respectively. As shown in the current individual view caption 402, the current view of the scheduling coordination software is configured for Dr. Brown. This is shown in the current individual view caption 402. The scheduling coordination software also shows the other radiologists working for this shift—Dr. Green and Dr. White—in the staff for current shift view caption 408. The caption 408 also indicates a letter “B”,“G”, and “W” next to the three staffed radiologists. The first state “B” may indicate to Dr. Brown that he may efficiently perform John Smith's examination. The second state “G” may indicate that Dr, Green may perform the examination of Peter Lee more efficiently. The third state “W” may indicate that Dr. White may perform the examination of Rosa Perez more efficiently. The fourth state “###” may indicate that there may be no efficiency advantage among staffed radiologists for performing Melanie Jackson's examination.

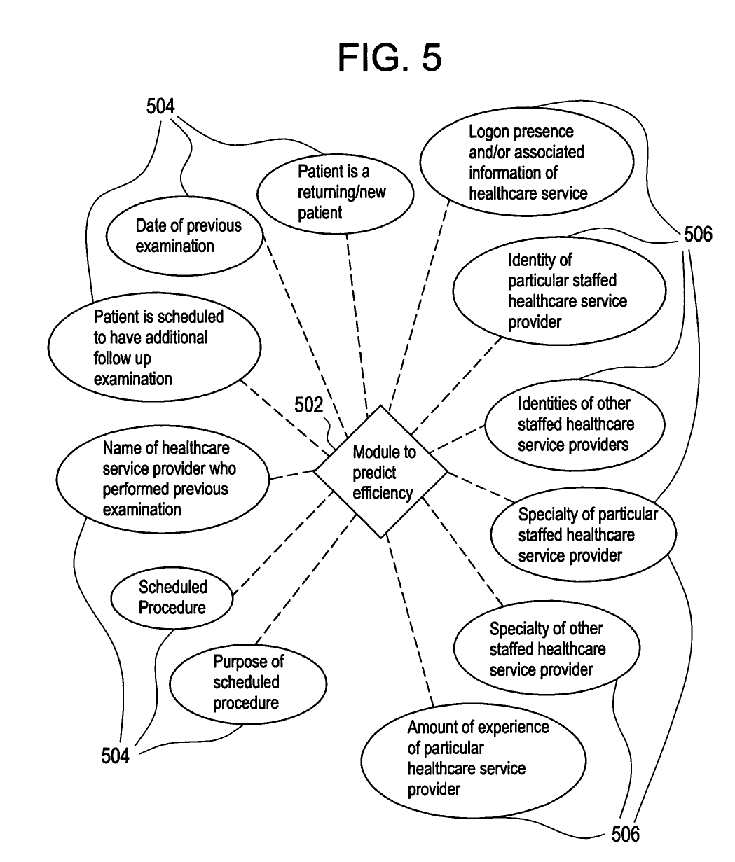

In order to predict efficiency of having a healthcare service provider perform an examination, a variety of information may be considered. FIG. 5 shows a block diagram of information 504, 506 useable by a prediction module 502 to predict an efficiency according to an embodiment of the present invention. A variety of information 504, 506 may be considered by a prediction module 502 that predicts an efficiency of having a particular healthcare service provider perform the scheduled examination. The prediction module 502 may be implemented with hardware, software, firmware, or a combination thereof. For example, the prediction module 502 may be executable on a PACS workstation 208. The prediction module 502 may be integrated into scheduling coordination software, for example. The prediction module 502 may include a plurality of sub-modules, which may be physically or conceptually separated from each other.

Some information 504 may be associated with a patient. For example, patient associated information 504 may include patient records, and/or patient scheduling information. Patient associated information 504 may include a wide variety of information. A few examples of patient associated information 504 include: patient is a returning/new patient; date of previous exam; patient is scheduled to have additional follow-up examination; name of healthcare service provider who participated in a previous examination; name of healthcare service provider who approved a previous examination; name of healthcare service provider associated with previous examination; name of scheduled procedure; purpose of scheduled procedure; modality of scheduled procedure; number of images in scheduled procedure. Patient associated information 504 may be communicated to prediction module 502 from PACS 106, RIS 104, or HIS 102.

Patient associated information 504 may also be communicated through protocols. For example, HL7 is a healthcare application protocol accredited as a standard by the American National Standards Institute (“ANSI”). The HL7 family of standards specify the implementation of interfaces between computer applications. The HL7 protocol defines message structures with various segments. Applications running on a PACS workstation may employ HL7 to communicate through PACS 106, RIS 104 and/or HIS 102 to exchange information with other applications. One segment of HL7 is called the Patient Visit (PV) segment. HL7 can communicate information corresponding to one or more patient visits—e.g. PV1, PV2 . . . , PVn. Each PV segment, corresponding to a previous patient visit, potentially includes a variety of information. A few examples include: admission type, prior patient location, attending doctor, referring doctor, consulting doctor, admitting doctor, visit number, hospital service, patient type, diet type, bed status, patient account and credit information, date/time of admission, and other healthcare provider information. Thus, through protocols such as HL7, a scheduling coordination software may receive or retrieve patient associated information 504. A RIS 104 may contain patient associated information 504, such as patient scheduling information. The RIS 104 may communicate the patient associated information 504 via a HL7 message. The patient associated information 504 may be communicated to a scheduling coordination software operating on a PACS 106, for example. The scheduling coordination software may update its database with the patient associated information 504, for example. The patient associated information 504 may be later used for determining an efficiency of a healthcare service provider.

Some information 506 may be associated with healthcare service providers. Healthcare service provider associated information 506 may include a variety of information. A few examples of healthcare service provider associated information 506 include: logon presence and associated information of a particular healthcare service provider; identity of a particular staffed healthcare service provider; identities of other staffed healthcare service providers; specialty of a particular healthcare service provider; specialties of other staffed healthcare service providers; amount of experience of a particular healthcare service provider; availability of a particular healthcare service provider. Healthcare service provider associated information 506 may be communicated to prediction module 502 from PACS 106, RIS 104, or HIS 102. For example, a logon presence of a healthcare service provider may be detectable, and communicated to prediction module 502. A healthcare service provider may logon, for example, on a PACS workstation 208, and the prediction module 502 may have a process running on the PACS workstation 208 that is capable of detecting a logon presence. Additional information may also be communicated with a logon presence. For example, a PACS 106 or a PACS workstation 208 may store a database or registry that associates additional information with a healthcare service provider. When a particular healthcare service provider logs on to a PACS workstation 208, the associated information 506 may be communicated to the prediction module 502. Healthcare service provider associated information 506 may be stored in a departmental information system such as RIS 104. A PACS 106 may retrieve healthcare service provider associated information 506 from an RIS 104, or other source, via a software request sent over the network.

Prediction module 502 may be able to evaluate patient associated information 504 and healthcare service provider information 506 to predict an efficiency of having a healthcare service provider perform the patient's scheduled examination. For example, prediction module 502 may be able to evaluate the purpose of a patient's scheduled procedure and the specialization of a particular healthcare service provider to predict whether the particular healthcare service provider may efficiently perform the examination. If, in this example, the scheduled procedure is for mammography, and the healthcare service provider has a specialization in breast imaging, then prediction module 502 may predict that the particular provider may efficiently perform this patient's scheduled examination. The efficiency predicted by prediction module 502 may be useable to adjust a state of a coordination indicator 410.

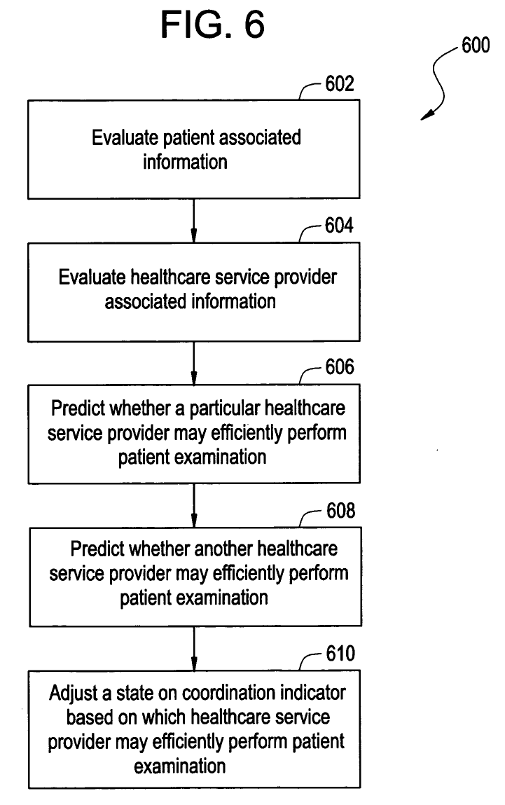

FIG. 6 shows a flowchart of a method 600 for adjusting a state of coordination indicator 410 in accordance with an embodiment of the present invention. Method 600 may be performed with hardware, software, firmware, or a mix thereof, for example. Method 600 may be performable, for example, by an application program or a set of instructions on a computer readable medium. Method 600 may be at least partially performed by scheduling coordination software.

At step 602, patient associated information 504 may be evaluated. As discussed, patient associated information 504 may include a variety of information associated with patient records and/or patient scheduling. Certain patient associated information 504 may be more relevant than other of patient associated information 504. For example, information related to a type of scheduled procedure may be more important than insurance billing information, although both may be patient associated information. The type and amount of patient associated information 504 evaluated at step 602 may vary based on system design, software design, and/or user preferences. For example, a user or a system administrator may determine that certain patient associated information 504 should be evaluated at step 602. Some patient associated information 504, such as general patient information, may be stored in HIS 102. Other patient associated information 504, such as radiological-specific patient information, may be stored in RIS 104. The patient associated information 504 may be stored in the information systems' databases, such as a HIS 102 database or a RIS 104 database, for example. Patient associated information 504 may be communicated to a module to predict efficiency 502 (shown in FIG. 5), which may evaluate patient associated information 504.

At step 604, healthcare service provider associated information 506 may be evaluated. As discussed, healthcare service provider information 506 may include a variety of information. Healthcare service provider information 506 may correspond to one or more healthcare service providers who are scheduled to work during a particular shift. Certain healthcare service provider associated information 506 may be more relevant than other of healthcare service provider associated information 506. For example, information related to a healthcare service provider's specialization may be more important than a healthcare service provider's age, although both may be healthcare service provider associated information 506. The type and amount of healthcare service provider associated information 506 evaluated at step 604 may vary based on system design, software design, user preferences, and/or other criteria, for example. For example, a user or a system administrator may determine that certain healthcare service provider associated information 506 should be evaluated at step 604. A departmental information system such as RIS, or a general information system such as a HIS, could store healthcare service provider associated information 506 in its database. The healthcare service provider associated information 506 may be stored in the information systems' databases, such as a HIS 102 database or a RIS 104 database, for example. Healthcare service provider associated information 506 may be communicated to a module to predict efficiency 502 (shown in FIG. 5), which may evaluate healthcare service provider associated information 506.

At step 606, method 600 may predict whether a particular healthcare service provider may efficiently perform an examination. A particular healthcare service provider may correspond to the service provider referenced by the current individual view caption 402 (shown in FIG. 4). For example, a particular healthcare service provider may be an individual logged into a PACS workstation 208. The prediction at step 606 may be performed by comparing the evaluated patient associated information 504 from step 602 with the evaluated healthcare service provider associated information 506 from step 604. The prediction may look for matches between the particular healthcare service provider information 506 and the patient associated information 504. For example, the prediction may assess if a scheduled procedure falls within a particular healthcare service provider's area(s) of specialization. As another example, the prediction at step 606 may assess whether the particular healthcare service provider has approved previous examinations of the scheduled patient. A previous examination may have been approved by a healthcare service provider if, for example, the provider signed off on the previous examination or otherwise attributed his/her authority in conjunction with the performance of the previous examination. The prediction may also weigh various factors in combination, based on rules of prioritization. The nature of the prediction at step 606 may vary based on system design, software design, and/or user preferences.

At step 608, method 600 may predict whether another healthcare service provider, aside from the particular healthcare service provider of step 606, may efficiently perform an examination on a scheduled patient. Another healthcare service provider may correspond to the additional service providers who are referenced by the staff for current shift view caption 408 (shown in FIG. 4). For example, another healthcare service provider may be an individual scheduled to work during the same shift as the particular healthcare service provider. The prediction at step 608 may be performed by comparing the evaluated patient associated information 504 from step 602 with the evaluated healthcare service provider associated information 506 from step 604. The prediction may look for matches between the particular healthcare service provider information 506 and the patient associated information 504. For example, the prediction may assess if a scheduled procedure falls within another healthcare service provider's area(s) of specialization. As another example, the prediction at step 608 may assess whether another healthcare service provider has approved previous examinations of the scheduled patient. The prediction may also weigh various factors in combination, based on rules of prioritization. The nature of the prediction at step 608 may vary based on system design, software design, user preferences, and/or other criteria, for example.

As a result of step(s) 606 and/or 608, there may be a predicted efficiency of having a healthcare service provider perform the scheduled examination. It should be understood that a predicted efficiency need not be quantifiable. Instead, a predicted efficiency is a broad term indicating that there may be some basis for predicting that a healthcare service provider may perform an examination efficiently. For example, there may be some basis for a predicted efficiency if a radiologist is chosen to perform an examination within his or her specialty. As another example, there may be some basis for a predicted efficiency if a radiologist has recently examined the same patient for the same type of imaging procedure.

At step 610, a state of coordination indicator 410 may be adjusted based on the predicted efficiency of step(s) 606 and/or 608. The state of coordination indicator 410 may be adjusted to indicate to a healthcare service provider that he or she may be able to efficiently perform a patient examination. For example, if a particular healthcare service provider may be predicted to be able to perform a patient examination efficiently in step 606, coordination indicator may be adjusted to indicate to the particular healthcare service provider that he or she may efficiently perform this patient's exam. It may be possible to adjust a state of coordination indicator 410 to represent that multiple healthcare service providers may be able to efficiently perform an examination. The result of step 610 may vary based on a variety of factors. For example, looking back at FIG. 4, the coordination indicator 410 state may vary based on which service provider may be indicated in the current individual view caption 402. As another example, the nature of the prediction at step 608 may vary based on system design, software design, and/or user preferences.

Steps of method 600 may be performed in a different order. Steps may also be performed simultaneously, or in parallel. Additionally, some steps, such as step 608 for example, may be omitted.

Method 600 may be performed in the following manner, for example. Starting at step 602, patient John Smith's information may be evaluated to determine if he is a returning patient. During John Smith's prior visit, he had an imaging procedure that was examined by Dr. Brown, who also signed off on the examination. This previous imaging procedure was performed one month ago. Continuing to step 604, Dr. Brown, Dr. Green, and Dr. White are the radiologists scheduled during the shift when John Smith's examination is to take place. None of the scheduled radiologists have a particular specialization or sub-specialization. Continuing to step 606, method 600 predicts that a particular radiologist, Dr. Brown, may efficiently perform John Smith's examination. This prediction was made because Dr. Brown has examined this patient within the last month, and may have already acquired a degree of familiarity with the patient and the medical issues presented. Continuing to step 608, method 600 predicts that neither Dr. Green, nor Dr. White have an identifiable efficiency advantage with respect to John Smith's examination. Neither of Drs. Green or White have previously examined John Smith, and neither have an identified specialization that may be useful to this exam. Continuing to step 610, the state of coordination indicator 410 is adjusted to “B”, which corresponds to Dr. Brown. Thus, coordination indicator 410 has been adjusted to communicate to Dr. Brown, or to Dr. Green and Dr. White, that Dr. Brown may efficiently perform John Smith's examination.

Portions of method 600 and/or the prediction module to predict efficiency 502 (shown in FIG. 5) may be used in additional ways. For example, method 600 may be used to automatically coordinate scheduling between a patient and a healthcare service provider. Once a predicted efficiency is determined at steps 606 and/or 608, scheduling coordination software may automatically schedule an incoming patient to be examined by an appropriate healthcare service provider. Additionally, scheduling coordination software may provide a notification corresponding to a scheduling coordination. The substance of the notification may be communicated to a variety of parties. For example, the notification may be communicated to a healthcare service provider, a patient, a healthcare service administrator, and/or an insurance provider or administrator. The substance of the notification may also be communicated to other computer systems, software applications, and databases. For example, after scheduling coordination software makes a scheduling decision, a notification may be communicated to global scheduling software applications and databases in a HIS and/or RIS. It should be understood that it may be necessary to communicate a notification to digital systems in order to have an intended recipient receive the notification.

Portions of method 600 and/or the prediction module to predict efficiency 502 (shown in FIG. 5) may also be employed to further assist the scheduled healthcare service provider in anticipation of the upcoming examination. For example, radiologists may perform examinations on a PACS workstation. Each radiologist may have his or her own PACS workstation. Once a scheduling coordination decision has been made, the appropriate patient images and/or records may be routed to the scheduled radiologist's PACS workstation, and stored locally. For example, patient images and/or records may be routed to a PACS workstation based on user login, or a Thus, the radiologist may be able to rapidly access potentially pertinent patient information.

Thus, embodiments of the present invention provide methods and systems that communicate helpful information to a healthcare service provider who is deciding whether to perform an examination of a scheduled patient. Additionally, embodiments of the present invention provide methods and systems that enable a healthcare service provider to select efficiently performable examinations. Moreover, embodiments of the present invention provide methods and systems that enable a healthcare service provider to defer an examination to another healthcare service provider who may perform the examination efficiently.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. For example, features may be implemented with software, hardware, firmware, or a mix thereof. Therefore, it is intended that the invention not be limited to the particular embodiment disclosed, but that the invention will include all embodiments falling within the scope of the appended claims.

1. A method of coordinating scheduling comprising:
evaluating patient associated information corresponding to a patient having a scheduled examination;
evaluating healthcare service provider associated information corresponding to at least one healthcare service provider;
predicting an efficiency for one of said at least one healthcare service provider performing said scheduled examination based at least in part on said patient associated information and said healthcare service provider associated information; and
adjusting a state of a coordination indicator based at least in part on said efficiency.

2. The method of claim 1, wherein said healthcare service provider comprises a radiologist.

3. The method of claim 1, wherein said healthcare service provider associated information comprises at least one of: a logon presence of said at least one healthcare service provider; an identity of a staffed said at least one healthcare service provider; a specialty of said staffed said at least one healthcare service provider; an availability of said at least one healthcare service provider; and an experience of said staffed said at least one healthcare service provider.

4. The method of claim 1, whereby at least a portion of the method is performable by a Picture Archiving and Communications System.

5. The method of claim 1, wherein said efficiency is based at least in part on a prior examination of said patient having been performed by an available one of said at least one healthcare service provider.

6. The method of claim 1, wherein said patient associated information comprises at least one of: information that said patient is a returning patient; a date of a prior examination of said patient; information that said patient is scheduled to have an additional follow-up examination; a name of a healthcare service provider who participated in said prior examination; a name of a healthcare service provider who approved said prior examination; a name of healthcare service provider associated with said prior examination; a type of said scheduled examination; a purpose of said scheduled examination; a modality of said scheduled examination; and a number of images in said scheduled examination.

7. The method of claim 1, wherein said state of said coordination indicator corresponds to at least one of: a suggestion for one of said at least one healthcare service provider to perform said scheduled examination; a suggestion for other of said at least one healthcare service provider to perform said scheduled examination; and no suggestion for any of said at least one healthcare service provider to perform said scheduled examination.

8. The method of claim 1 further comprising assigning said at least one healthcare service provider to perform said scheduled examination based at least in part on said efficiency.

9. The method of claim 1 further comprising notifying at least one party based at least in part on said efficiency, said at least one party comprising at least one of: said patient; said at least one healthcare service provider; a healthcare service administrator; an insurance provider; an insurance administrator.

10. A system for coordinating patient examination scheduling comprising:
patient associated information corresponding to a patient having a scheduled examination;
healthcare service provider associated information corresponding to at least one healthcare service provider scheduled to work at the time of said scheduled examination;
a prediction module capable of receiving said patient associated information and said healthcare service provider associated information, said module capable of predicting an efficiency of having one of said at least one healthcare service provider perform said scheduled examination based at least in part on said patient associated information and said healthcare service provider associated information; and
an application software capable of displaying a coordination indicator based at least in part on said efficiency.

11. The system of claim 10, wherein said healthcare service provider comprises a radiologist.

12. The system of claim 10, wherein said efficiency is based at least in part on a correspondence between said patient associated information and said healthcare service provider associated information.

13. The system of claim 10, wherein said healthcare service provider associated information comprises at least one of: an identity of an available healthcare service provider, and said patient associated information comprises a prior examination approved by said available healthcare service provider.

14. The system of claim 13, wherein said available healthcare service provider approved said prior examination.

15. The system of claim 10, wherein at least a portion of said prediction module is executable on a Picture Archiving and Communication System.

16. The system of claim 10, wherein said coordination indicator corresponds to at least one of: a suggestion for one of said at least one healthcare service provider to perform said scheduled examination; a suggestion for other of said at least one available healthcare service provider to perform said at least one scheduled examination; and no suggestion for any of said at least one healthcare service provider to perform said scheduled examination.

17. The system of claim 10, wherein said healthcare service provider associated information comprises at least one of: a logon presence of said at least one healthcare service provider; an identity of a staffed said at least one healthcare service provider; a specialty of said staffed said at least one healthcare service provider; an availability of said at least one healthcare service provider; and an experience of said staffed said at least one healthcare service provider.

18. The system of claim 10, wherein said patient associated information comprises at least one of: information that said patient is a returning patient; a date of a prior examination of said patient; information that said patient is scheduled for an additional follow-up examination; a name of a healthcare service provider who participated in said prior examination; a name of a healthcare service provider who approved said prior examination; a name of healthcare service provider associated with said prior examination; a type of said scheduled examination; a purpose of said scheduled examination; a modality of said scheduled examination; and a number of images in said scheduled examination.

19. A computer-readable storage medium including a set of instructions for a computer, the set of instructions comprising:
at least one evaluation routine for evaluating patient associated information corresponding to a patient having a scheduled examination and for evaluating healthcare service provider associated information corresponding to at least one healthcare service provider;
a prediction routine for predicting an efficiency for one of said at least one healthcare service provider performing said scheduled examination based at least in part on said patient associated information and said healthcare service provider associated information; and
an adjustment routine for adjusting a state of a coordination indicator based at least in part on said efficiency.

20. The set of instructions of claim 19, wherein said healthcare service provider comprises a radiologist.

21. The set of instructions of claim 19, wherein said healthcare service provider associated information comprises at least one of: a logon presence of said at least one healthcare service provider on a Picture Archiving and Communications System; an identity of a staffed said at least one healthcare service provider; a specialty of said staffed said at least one healthcare service provider; an availability of said at least one healthcare service provider; and an experience of said staffed said at least one healthcare service provider.

22. The set of instructions of claim 19, wherein said efficiency is based at least in part on a prior examination of said patient having been performed by an available one of said at least one healthcare service provider.

23. The set of instructions of claim 19, wherein said state of said coordination indicator corresponds to at least one of: a suggestion for one of said at least one healthcare service provider to perform said scheduled examination; a suggestion for other of said at least one healthcare service provider to perform said scheduled examination; and no suggestion for any of said at least one healthcare service provider to perform said scheduled examination.

24. The set of instructions of claim 19, wherein said patient associated information comprises at least one of: information that said patient is a returning patient; a date of a prior examination of said patient; information that said patient is scheduled for an additional follow-up examination; a name of a healthcare service provider who participated in said prior examination; a name of a healthcare service provider who approved said prior examination; a name of healthcare service provider associated with said prior examination; a type of said scheduled examination; a purpose of said scheduled examination; a modality of said scheduled examination; and a number of images in said scheduled examination.
